(19) United States
(12) Patent Application Publication
(10) Pub. No.: US 2024/0277297 A1
(43) Pub. Date: Aug. 22, 2024

(54) FALL RISK ASSESSMENT DEVICE

(71) Applicant: The University of Hong Kong, Hong Kong (CN)

(72) Inventors: Ning XI, Hong Kong (CN); Song WANG, Hong Kong (CN); Jiaming CHEN, Hong Kong (CN); Kehan ZOU, Hong Kong (CN); Weiqun LOU, Hong Kong (CN); Xin MA, Hong Kong (CN)

(73) Assignee: The University of Hong Kong, Hong Kong (CN)

(21) Appl. No.: 18/571,200

(22) PCT Filed: Jun. 13, 2022
(86) PCT No.: PCT/CN2022/098354

§ 371 Date: Dec. 18, 2023

Related U.S. Application Data
(60) Provisional application No. 63/210,596, filed on Jun. 15, 2021.

(51) Int. Cl. A61B 5/00; A61B 5/11

(52) U.S. Cl. CPC A61B 5/7275; A61B 5/1117; A61B 5/4023; A61B 5/7267; A61B 2576/00

(57) ABSTRACT

A human balance sensor for assessing the risk of the user falling includes a transparent glass plate, a latex sheet located on the top surface of the glass plate, a light source located so as to inject light into edge of the glass plate and a high resolution camera located below the glass plate so as to capture light diffused from the glass plate when pressure is applied thereto by the user's foot. Based on the principle of Frustrated Total Internal Reflection (FTIR), when user stands with his feet on the glass plate a condition of total internal reflection is eliminated at pressure locations due to the pressure from the feet, and diffused light passes from the bottom surface of the glass plate and forms a haptic image of the contact area of the feet which can be analyzed over time to determine the user's ability to balance, and hence their risk of a fall.

This application is a U.S. National Stage Application under 35 U.S.C. § 371 of International Patent Application No. PCT/CN2022/098354, filed Jun. 13, 2022, and claims the benefit of priority under 35 U.S.C. Section 119(e) of U.S. provisional patent application U.S. 63/210,596 filed on Jun. 15, 2021, the entire content of which is incorporated by reference for all purpose. The International Application was published in English on Dec. 22, 2022 as International Publication No. WO 2022/262669 A1.

### TECHNICAL FIELD

The present invention relates to assessing the risk of falling for elderly people and, more particularly, to a device upon which a user can step and which measures the dynamic force distribution on the user's feet to calculate the risk of the user falling.

### BACKGROUND

Falls are major threats to the health and independent living of the elderly. It is estimated that 10% of the falls by the elderly are associated with fractures, and some can lead to head injuries and deaths. Falls and their associated injuries, such as hip fractures, are risk factors for placement in nursing homes [MT1997]. Even minor falls can lead to substantial functional impairment of mobility and the daily activities of living in the elderly. They can trigger a negative domino effect leading to complications such as pneumonia, thromboembolism, loss of autonomy, disability, anxiety, depression and an impaired quality of life in the individual, and a burden on the family. Falls in the elderly are costly to the health care system because they often require accident and emergency services, as well as prolonged hospitalization, procedures, operations and rehabilitation services. The burden of falls on the society will increase with the aging population.

Since an average of 20% of the elderly population have accidental falls annually, if at least 10% of them can be warned of an imminent fall risk so they can take proper precautions, great harm to them can be prevented. In particular, severe injuries such as fractures, head injuries and deaths could be reduced from 10% to 3% by taking proper preventive measures. However, current assessment tools for falls and balance require the presence of a clinician for administration of a test and interpretation of the results. Since individuals do not have a practical and objective way to assess their own daily fall risk, they may under-estimate or overestimate their risk of falling. While under-estimation leads to unsafe behaviors and increased falls, over-estimation is also problematic due to creation of an unfounded fear of falling and its downstream effects such as restricted physical activity, social isolation and functional loss.

Balance ability assessment relies on a specific procedure or method that can analyze human balance ability either quantitatively or qualitatively. Currently, there are a wide range of different methods for human balance ability assessment. These can be divided into three categories: observation, scale, and balance testing devices.

The simplest and most typically used method is observation, such as the Romberg test [FB1982, YA2011], one-legged stance test (OLST) [TM2009], and postural stress test [JC1990], etc. In the Romberg test, subjects close their eyes, stand on both feet, and raise their arms forward.

An evaluation person (evaluator) will then give a balance ability assessment based on the degree of body sway. Similar to the Romberg test, in an OLST, the subject instead stands on one leg. The postural stress test is clinically applicable and is used to obtain quantitative measurements. In this method, a destabilizing force is applied to the subject's waist. Balance ability is evaluated based on the subject's ability to remain standing upright.

A more elaborate method is a scale, which includes the Berg balance test [SM2008], Tinetti test [SK2006], and Timed Up and Go test (TUG) [TS2002], etc. The Tinetti test has also been widely used in balance ability assessment and fall prediction for the elderly. In this method, an evaluator will score the subject's performance in a series of different tasks.

The first balance testing device method was introduced by Yuriy V. Terekhov [YT1976] in 1976 and is called stabilometry. This measures the mechanical oscillation of a subject's center of gravity and converts it into electronic signals. A computer is then used to analyze the frequency, amplitude, and duration of oscillation to evaluate the subject's balance ability. Over the years, this method has been improved and developed into different versions, but the basic principle remains the same; they all consist of a pressure test board, computer, and specialized analysis software (see FIG. 1).

Balance testing devices have even been adapted for recreational use. For example, the Nintendo Wii Balance Board [RC2010] (see FIGS. 2A and 2B), which uses Bluetooth technology and contains four pressure sensors, one on each corner, to measure the user's center of pressure under each foot. Similar to the Wii Balance Board is the Intec Action Board and the GameOn Compatible Balance Board.

Fall risk assessment is used to determine if a subject has a low, moderate, or high risk of falling. Mostly performed on older adults, it commonly includes an initial screening, then completion of a set of tasks known as fall assessment tools. Initial screening comprises a series of questions about the subject's overall health, and whether they have a history of falls or problems with balance, standing, or walking; while the fall assessment tools test a subject's strength, balance, and gait.

Initial screening questions include: “Have you fallen in the past year?”; “Do you feel unsteady when standing or walking?”; and “Are you worried about falling?” There are a number of questionnaires that can be used for screening, such as the Patient Fall Questionnaire [NR1984] and the Fall Assessment Questionnaire [LR1993].

Fall assessment tools include the aforementioned TUG test [TS2002], 30-Second Chair Stand Test [KJ2015], and 4-Stage Balance Test [JG2017], etc. In the TUG test, the subject starts in a chair, stands up, then walks approximately 10 feet at a regular pace while the health care provider checks the subject's gait. The 30-Second Chair Stand Test checks strength and balance. First, the subject sits in a chair with their arms crossed over their chest. They then repeat standing up and sitting down for 30 seconds while the health care provider counts how many times this is performed. The 4-Stage Balance Test checks how well a subject can maintain their balance. The subject stands in four different positions, holding each one for 10 seconds. In the first position, the subject stands with their feet side by side. In the second, the subject moves one foot halfway forward. In the third, the subject moves one foot fully in front of the other, so that the toes touch the heel of the other foot. In the fourth position, the subject stands on only one foot. There are many other similar fall assessment tools, such as the Berg Balance Test [KB1989], Elderly Fall Screening Test [JC1998], Dynamic Gait Index [SW2000], and Tinetti Performance Oriented Motility Test [MT1986].

There are also scale methods for fall assessment, such as the Gait Abnormality Rating Scale (GMRS) [LW1990, JV1996] and Morse Fall Scale [JM1989]. GMRS, for example, includes variables that are intended to provide a description of test subject's gait associated with an increased risk of falling, such as step and arm movements, guardedness, weaving, waddling, staggering, percentage of time in the swing phase of the gait cycle, foot contact, hip range of motion, knee range of motion, elbow extension, shoulder extension, shoulder abduction, arm-heel-strike synchrony, head held forward, shoulder held elevated and upper trunk flexed forward.

Among the various human balance ability assessment methods, because an evaluator is required for both the observation and scale methods, balance ability evaluation is subjective. At the same time, fall assessment tools also require a health care provider to administer the assessment, meaning that the results are also subjective. As such, because no evaluator or health care provider is needed, a balance tester is much more objective. While the tread force detector in balance testers mostly relies on an array of electronic force sensors (see FIG. 3), due to its structure, the obtained tread force distribution resolution is very low. Moreover, the equipment is costly. Hence, there currently lacks a method that can objectively evaluate human balance ability with a high degree of accuracy and at a low cost.

### SUMMARY OF THE INVENTION

In one aspect of the present invention, there is provided a human balance sensor for assessing the risk of the user falling, comprising:
a transparent glass plate with flat upper and lower surfaces, and having a refractive index larger than that of air;
a latex sheet located on the top surface of the glass plate, during operation a foot of the standing user is placed on top of the latex sheet;
a light source located so as to inject light into the glass plate from its edge;
a high resolution camera located below the lower surface of the glass plate so as to capture light diffused from the glass plate when pressure is applied to the glass plate by the user's foot; and
whereby, based on the principle of Frustrated Total Internal Reflection (FTIR), when user stands with his foot on the glass plate (a) the latex sheet is pressed onto the upper surface of the glass plate, (b) a condition of total internal reflection is eliminated at the pressure locations due to the foot, and (c) diffused reflection of the light passes from the bottom surface of the glass plate and is focused onto an image plane of the camera to form a haptic image of the contact area of the foot with different pixel intensities based on different pressures from the foot at different locations on the glass plate.

In a preferred embodiment, the light given by the light source is invisible or more preferably an infrared light. The light source may be any light-emitting device that can give an invisible light (i.e. not visible by human naked eyes) at a fixed wavelength, particularly give infrared light at a fixed wavelength. It is advantageous to the assessment because it can minimize the noise detected during the entire process, so as to improve the accuracy of the measurement.

In another embodiment, the human balance sensor further comprises a wave guide or an optical wave guide to block unwanted light from entering the transparent glass plate or minimize the unwanted effect caused by noise. This could improve the accuracy in assessment.

In another aspect of the invention, there is provided a human balance sensor for assessing the risk of the user falling, comprising:
a housing;
two transparent glass plates with flat upper and lower surfaces, and having refractive indices larger than that of air, said glass plates being located side-by-side on a top of the housing and spaced from each other by about the spacing of the feet of a standing human;
latex sheets, one located on the top surface of each of the glass plates, during operation the feet of the standing user are placed on top of the respective latex sheets;
a light source located so as to inject light into each of the glass plates from their edges;
a high resolution camera located below the lower surface of the glass plates so as to capture light diffused from the glass plates when pressure is applied to the glass plates; and
whereby, based on the principle of Frustrated Total Internal Reflection (FTIR), when user stands with his feet on the glass plates (a) the latex sheets are pressed onto the upper surfaces of the respective glass plates, (b) a condition of total internal reflection is eliminated at the pressure locations due to the feet, and (c) diffused reflection of the light passes from the bottom surfaces of the glass plates and is focused onto an image plane of the camera to form a haptic image of the contact area of the feet with different pixel intensities based on different pressures from the feet at different locations on the glass plates.

Similarly, the human balance sensor of this aspect may also comprise an invisible light source as described above, and an optical wave guide.

In a further aspect, the invention pertains to a method of using the human balance sensor as described herein to assess the risk of a user failing.

Compared to “A device for measuring plantar pressure under the sole of the foot”, R. P. Bettes and T. Duckworth, here are some major differences in hardware design:
(1) Light source: In the prior art, a visible light source was used. But the present invention uses infrared LED that has fixed wavelength. It can significantly reduce the noise in Total Internal Reflection (TIF) and improve the accuracy of the measurement.
(2) Glass wave guide: The glass in the prior art is a regular glass. But the present invention uses a glass wave guide that only allows the infrared wavelength light to go through and form TIF. It can completely block the light from the environment and make the measurement much more accurate. One can even make the measurement without a soft surface layer.
(3) Mirror and Camera: Since the present invention uses fixed wavelength infrared as a light source, the mirror as well as the camera are all for the same wavelength. This can reduce the noise by blocking ambient light and improve accuracy by using fixed wavelength light.

In order to be objective when assessing human balance ability, the present invention uses a comprehensive physical system for balance evaluation, called “Balance Sensor,” which has a special sensing unit for collecting force distribution information under human feet. The sensing unit does not rely on an electronic force sensor array, but instead operates on the optical principle of Frustrated Total Internal Reflection (FTIR). Widely used to develop haptic sensors for robots [HM1992, NN1990, SB1988_1, SB1988_2], the present invention extends the principle far beyond robotics and takes advantage of the abundant haptic information available and applies it to investigate human balance ability. Although a sensing unit based on FTIR has a much simpler structure than an electronic force sensor array, it is more sensitive and can reach much higher force distribution resolution. Furthermore, its fabrication cost is very low.

As the sensing unit of the Balance Sensor is based on an optical principle, the final signal collecting device is a camera. Force distribution under the feet is recorded in images; hence, force distribution variation information is encoded in a video format (see FIG. 4). When subjects try to balance themselves, the force distribution under their feet will change with time, although very slightly. Not only can the highly sensitive and high-resolution Balance Sensor detect such subtle variations, but the device can also analyze the variation process from the recorded video to evaluate human balance ability.

Once the collected data is in video format, cutting-edge computer vision and AI technology can be leveraged for analysis, thereby greatly enhancing the Balance Sensor's ability to obtain human balance information. This is another major advantage compared with traditional balance testing devices. If the data analysis results need to be discrete, then algorithms are used to map the original video data to these discrete results, which is a classification solution. One method is to extract certain features manually from the original videos and then set a rule-based algorithm to classify different videos or train a machine learning model to classify them. Another method is to simply use the original video data to train a deep learning algorithm such as a 3D convolutional neural network (CNN) to generate a classification model. If the data analysis results are continuous, then a functional relationship is established between the original video data and the final continuous results. When this is the case, it is a regression process. As with the previous method, features can be extracted manually and a regression model can be trained accordingly, or deep learning methods can be used to perform regression. In both scenarios, however, it is better to first compress the original video data to extract useful information and discard redundant information before analysis. This is because there is always a large volume of video data and it allows for the reduction of the model size and improved processing efficiency. In terms of data compression, there are multiple available methods, such as compressive sensing and autoencoding.

Alternatively, there is another option to analyze the Balance Sensor's data. Since the relationship between pressure value and pixel intensity is fixed, this pressure-pixel relationship can be calibrated through experiments, so as to transform the original images into real pressure distribution information. This calibration work has been completed. [SW2019, SW2020] With the real pressure distribution variation information, dynamic analysis of the human body can be conducted. Specifically, a dynamic model can be established for the specific motion of a human body during testing. This model comprises multiple differential equations associated with the pressure distribution variation process under the feet. With this information, a series of definite conditions can be set according to the physical properties of the human body, so that differential equations can be solved to obtain detailed body motion processes. A novel differential equation-solving algorithm has been developed based on a Generative Adversarial Tri (GAT) model, which is able to solve nonlinear differential equations with any feasible definite condition. With detailed body motion processes, further balance ability assessment or fall assessment can be realized.

There are two possible application scenarios. (1) To qualitatively identify whether the subject is normal, sick, or drunk. For example, it may be necessary to perform a physical examination of a patient, identify whether a subject has a specific neural disease, or use it to identify a drunk driver. (2) To quantitatively score the balance ability or fall likelihood of a subject. For example, as part of athlete selection, pilot selection, or during the falling assessment of an elderly patient.

### BRIEF SUMMARY OF THE DRAWINGS

The foregoing and other objects and advantages of the present invention will become more apparent when considered in connection with the following detailed description and appended drawings in which like designations denote like elements in the various views, and wherein:

FIG. 1 is a perspective view of a prior art typical balance tester being operated by a user;

FIG. 2A is a photograph of the top surface of a prior art Wii balance board and FIG. 2B is a photograph of the bottom surface thereof;

FIG. 3 is a photograph of a prior art tread force detector;

FIG. 4 illustrates a sequence of camera images of force distribution information;

FIG. 5 is a schematic illustration of the optical principle of Frustrated Total Internal Reflection (FTIR) utilized with the present invention;

FIG. 6 is a schematic diagram of a Balance Sensor according to the present invention;

FIG. 7A is an illustration of a user standing on the Balance Sensor of the present invention, FIG. 7B is a stick figure model of a back view of the human body of the user, and FIG. 7C is a stick figure model of a side view of the human body of the user;

FIG. 8 is an example of the pressure distribution under the feet of a user standing on the Balance Sensor of the present invention;

FIG. 9 is an illustration of a neural network structure useful with the present invention;

FIG. 10 is a flow chart of the Generative Adversarial Tri-model (GAT) model for solving differential equations;

FIG. 11 is a diagram of a regression model in fall assessment software;

FIG. 12 is a photograph of a Balance Sensor prototype according to the present invention;

FIG. 13 is an illustration of a graphical user interface for the Balance Sensor system of the present invention; and

FIGS. 14A-14J are graphs of the center of pressure (COP) over a 10 second period of five test subjects measured on the Balance Sensor of the present invention when the subjects are in a normal state (FIGS. 14A-14E) and when they have consumed a large amount of alcohol (FIGS. 14F-14J).

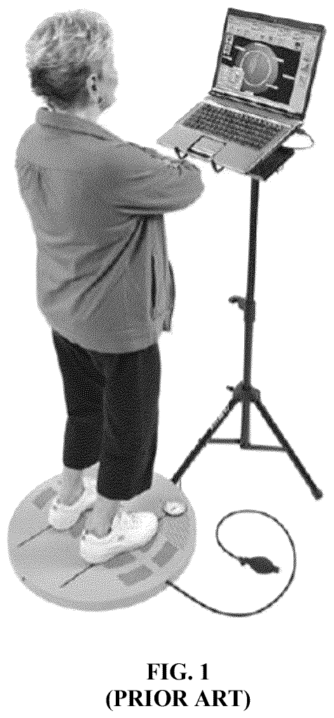

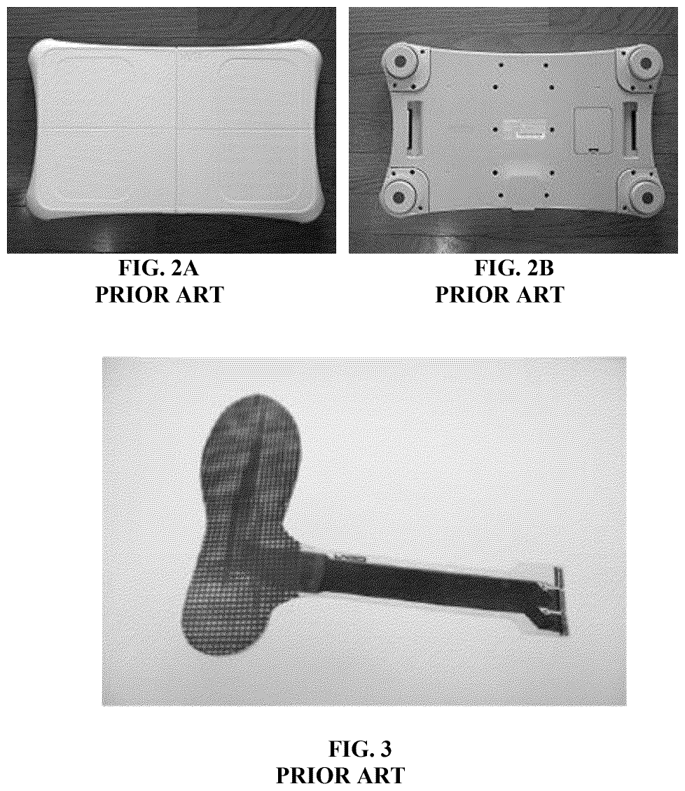

### DETAILED DESCRIPTION

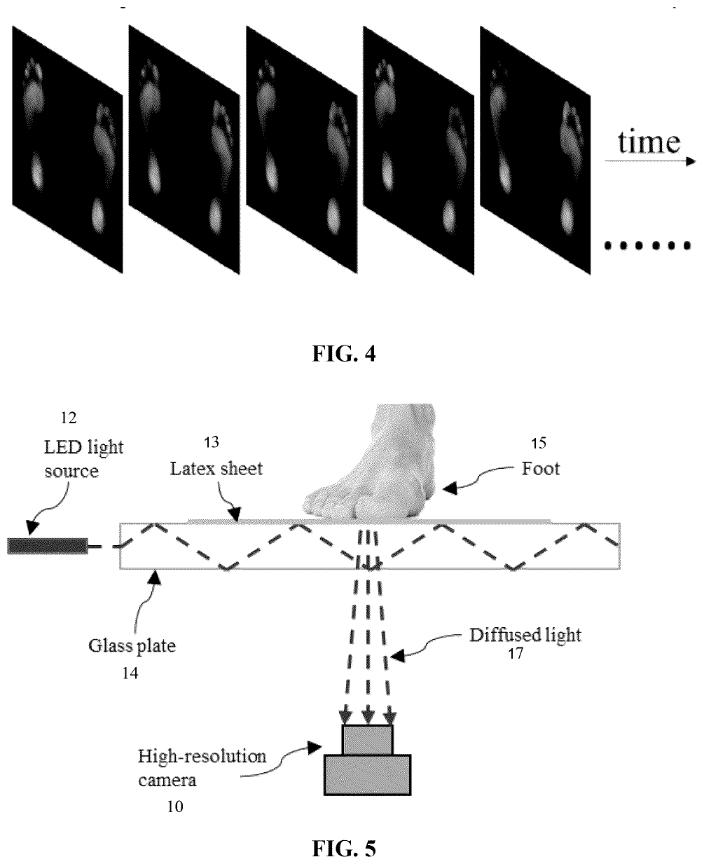

The major component of the Balance Sensor of the present invention is based on the principle of Frustrated Total Internal Reflection (FTIR), as shown in FIG. 5. This sensor mainly consists of a high resolution camera 10, LED light sources 12 and a thick transparent glass plate 14 with flat upper and lower surfaces. On the top surface of the glass plate, there is a piece 13 of latex sheet. The LED light source 12 injects light into the glass plate 14 from its edge. Because the refractive index of glass is larger than air, if nothing touches the glass surface, all the light will be reflected back into the glass plate and no light can be captured by the camera 10. However, when user stands on the glass plate with his foot 15 on the latex sheet 13, the latex sheet will be pressed onto the upper surface of the glass plate. At the contact area, the condition for total internal reflection will be destroyed and diffused reflection of the light happens instead. Part of the diffused light 17 will be captured by the camera 10 and focused on the image plane of the camera. Therefore, a haptic image of the contact area will be formed with different pixel intensities.

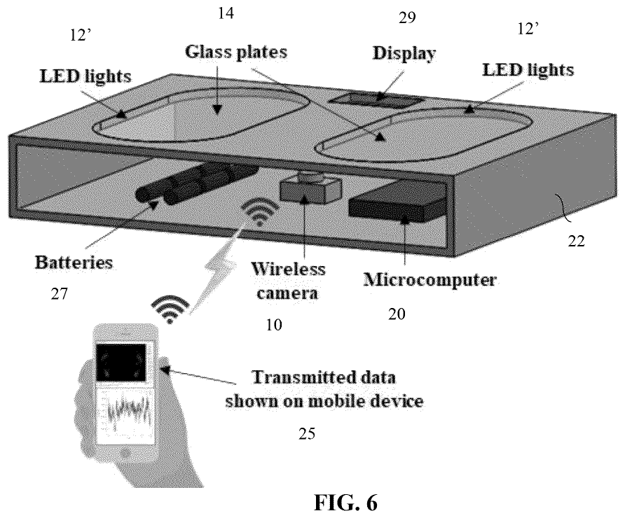

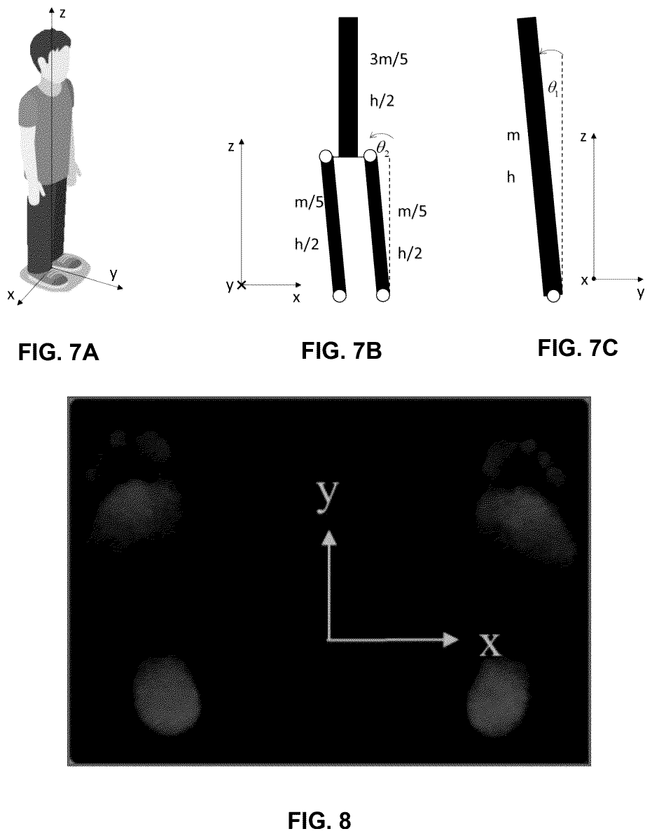

The different pixel intensities derive from different diffused light intensities at each point of contact. Different diffused light intensities are caused only by different contact pressure since the surface properties of the latex sheet and the glass plate are identical everywhere. Therefore, the haptic image captured by the camera is actually a force distribution image of the tread force under foot. Moreover, because the camera can record video with a high frame rate, the pressure distribution information can be recorded over time with a high frame rate. A schematic diagram of the Balance Sensor device of the present invention is shown in FIG. 6. It contains a sensing unit and a microprocessor 20. The sensing unit is roughly a box 22, which may have a black interior, with two glass plates 14 on top. On top of the two glass plates, there are two sheets of disposable latex. The two glass plates are both surrounded by LED light strips 12′, which may for example emit red light. At the bottom of the interior of the box, there is the camera 10. But because the peak tremor frequency that human limbs can reach is only around 10 Hz [JM1997], according to Shannon's sampling theorem, in order to retain the full information contained in original human body motions, the frame rate of the camera is 30 fps. The resolution is 1920×1440, which is much higher than currently existing balance testing devices available in the market and can meet the requirements for assessing human dynamic balancing. The LED lights and camera are operated according to FTIR principles by the microprocessor (microcomputer) 20. Also, the fall assessment results, i.e., the likelihood of a user to fall, which can be calculated in the microprocessor, are exhibited on a display 29 located on the top surface of the box. The camera may be wired to the microprocessor 20 or another remote computing device, or the camera may be wirelessly connected so the images generated by the camera can be transmitted to a remote display, e.g., a mobile device (e.g., an iPhone) 25. Power for the camera, lights and microprocessor is provided by batteries 27 As shown in FIG. 7 a coordinate system model can be built for a standing person in order to describe a person's dynamic balancing. A human body as shown in FIG. 7A can be simulated with 3 rigid bars hinged together as shown in FIG. 7B. When viewed from the back, the trunk and arms are considered as a single bar whose direction is always upright. The two legs are considered as two bars, which can swing in the x-z plane. The rotations of the two legs in the x-z plane are always the same. Viewing the model from the right side (FIG. 7C), the whole body can swing in the y-z plane. The rotation of the trunk and two legs are always the same. The mass of the human body is taken as m, with 3 m/5 for the trunk and m/5 for each leg. The height is h, with h/2 for each leg and the trunk. The whole model has two degrees of freedom (DoFs). The first degree of freedom is θ₁, indicating the rotation in the y-z plane, with the positive direction being anti-clockwise. The second degree of freedom is θ₂, indicating the rotation of both legs in the x-z plane, also with the positive direction being anti-clockwise. The trunk always stays upright. The pressure distribution p(x,y) under the user's feet as measured by the balance sensor is shown in FIG. 8. The dynamic model of the human body while standing can be built using the Lagrange equation, as shown in Eq1. There (x₀,y₀) is the coordinate of the middle point of the two ankles and (COPₓ,COP_{y}) is the coordinate of the center of pressure (COP) under the feet. The calculation method of (COP_{X},COP_{y}) is shown in Eq2. The value of (x₀y₀) can be approximated with the mean value of (COP_{X},COP_{y}) over a relatively long time.

$$\{ \begin{matrix}\frac{23}{60} \frac{{h}^{2}}{g} {\ddot{θ}}_{1} - \frac{31}{60} \frac{{h}^{2}}{g} {\dot{θ}}_{1} {\dot{θ}}_{2} {θ}_{2} - \frac{11}{20} h {θ}_{1} = {COP}_{y} - {y}_{0} \\ \frac{11}{60} \frac{{h}^{2}}{g} {\ddot{θ}}_{2} + \frac{31}{120} \frac{{h}^{2}}{g} {\dot{θ}}_{1}^{2} {θ}_{2} - \frac{2}{5} h {θ}_{2} = {COP}_{x} - {x}_{0}\end{matrix} \qquad ( Eq1 )$$

$$( {COP}_{x} , {COP}_{y} ) = ( \frac{\int \int p ( x , y ) xdxdy}{\int \int p ( x , y ) dxdy} , \frac{\int \int p ( x , y ) ydxdy}{\int \int p ( x , y ) dxdy} ) \qquad ( Eq2 )$$

Eq1 are nonlinear ordinary differential equations that have no analytical solution. Even though only a numerical solution is needed, the equations still lack initial conditions. However, other definite conditions can be exploited in order to solve Eq51. Since during experiments, the user or tester doesn't fall, θ₁ and θ₂ must always oscillate around 0. The angular velocities \dot{θ}₁ and \dot{θ}₂ also must always oscillate around 0. Since all of θ₁, θ₂, \dot{θ}₁, \dot{θ}₂ don't diverge, their integrals over the whole experimental period (0, T) are all considered to be 0. In this way, definite conditions are obtained as shown in Eq3.

$$\{ \begin{matrix}{\int }_{0}^{T} {θ}_{1} dt = 0 \\ {\int }_{0}^{T} {θ}_{2} dt = 0 \\ {\int }_{0}^{T} {\dot{θ}}_{1} dt = 0 \\ {\int }_{0}^{T} {\dot{θ}}_{2} dt = 0\end{matrix} \qquad ( Eq3 )$$

The present invention uses a novel method to solve the ordinary differential equation, the so called Generative Adversarial Tri-model (GAT) model. The GAT method combines an analytical approach with a neuro network to numerically solve nonlinear ordinary differential equations with non-initial conditions such as Eq3 as follows:

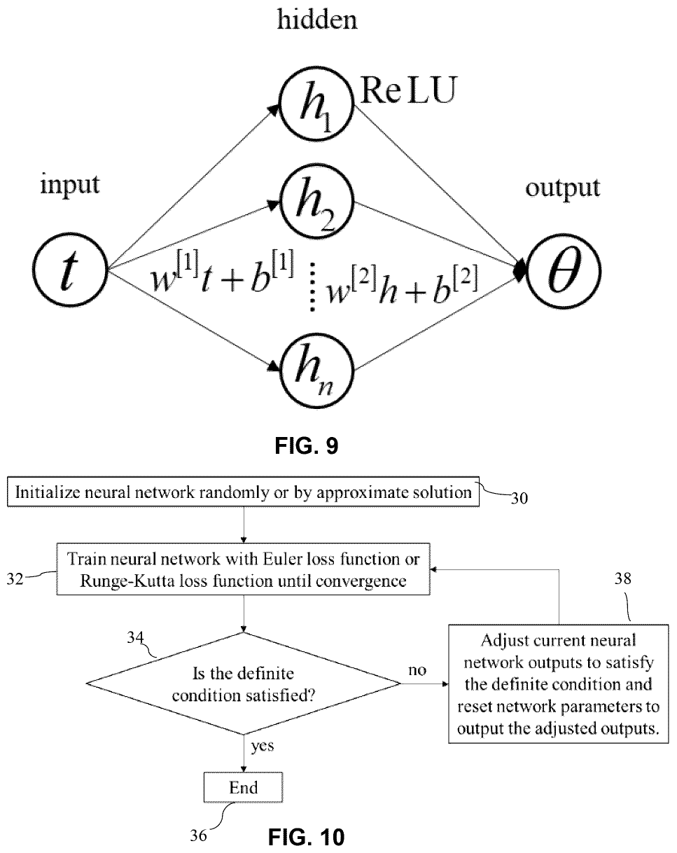

First Eq1 is transformed into 4 first-order differential equations, as shown in Eq4, where u₁=θ₁, u₂=θ₂, u₃=\dot{θ}₁, u₄=\dot{θ}₂ Specifically, four neural networks are used to represent θ₁|(t), θ₂(t), \dot{θ}₁(t), \dot{θ}₂(t), respectively. Their network structures are the same, as shown in FIG. 9. The number of hidden nodes is equal to the T*frame rate of the camera. This network can reproduce any numerical solutions of Eq4 according to a simple procedure. The loss function value is the mean square residual of Eq4 at all discrete numerical points, where the derivatives are approximated by the Euler manner or the Runge-Kutta manner.

$$\{ \begin{matrix}{\dot{u}}_{1} = {u}_{3} \\ {\dot{u}}_{2} = {u}_{4} \\ {\dot{u}}_{3} = \frac{31}{23} {u}_{2} {u}_{3} {u}_{4} + \frac{33 g}{23 h} {u}_{1} + \frac{60 g}{23 {h}^{2}} ( {COP}_{y} - {y}_{0} ) \\ {\dot{u}}_{4} = - \frac{31}{22} {u}_{2} {u}_{3}^{2} + \frac{24 g}{11 h} {u}_{2} + \frac{60 g}{11 {h}^{2}} ( {COP}_{x} - {x}_{0} )\end{matrix} \qquad ( Eq4 )$$

The flow chart of the GAT model is shown in FIG. 10. As a first step 30 the neural network is initialized randomly or by an approximate solution. Then the GAT model is trained (step 32) until convergence to obtain the numerical solutions of Eq4. In particular, the neural network is trained with the Euler loss function or the Runge-Kutta loss function until convergence. In order to determine this, a decision is made at step 34 as to whether the definite condition is satisfied. If so, the process ends at step 36. If not, the process proceeds to step 38 where the current neural network has its outputs adjusted to satisfy the definite condition and the network parameters are reset so as to output the adjusted values. The new network is trained at step 32 and the process repeats until the definite condition is satisfied.

Furthermore, an approximate solution is worked out and then this approximate solution is used as the first initialization of the GAT model. In this way, the convergence of the HAN model is made faster and better. Specifically, for Eq4, the nonlinear terms in the equations are first discarded so that Eq4 can be converted into linear differential equations Eq5. For Eq5, since it is linear, its numerical solutions can be worked out through the finite difference method with the help of definite conditions Eq3. Then the numerical solutions of Eq5 are used as the first initialization of the HAN model. This greatly accelerates the convergence of the HAN model. The multiple differential equations solved by this method are associated with the pressure distribution variation process under the feet of the user.

$$\{ \begin{matrix}{\dot{u}}_{1} = {u}_{3} \\ {\dot{u}}_{2} = {u}_{4} \\ {\dot{u}}_{3} = \frac{33 g}{23 h} {u}_{1} + \frac{60 g}{23 {h}^{2}} ( {COP}_{y} - {y}_{0} ) \\ {\dot{u}}_{4} = \frac{24 g}{11 h} {u}_{2} + \frac{60 g}{11 {h}^{2}} ( {COP}_{x} - {x}_{0} )\end{matrix} \qquad ( Eq5 )$$

There are lots of different coordinates of the center of pressure (COP)-based measurements that can be used to evaluate human balance ability or conduct fall assessment. Time-domain “distance” measures [TP1996] include mean distance of the COP from the origin, root mean square distance of the COP from the origin, total length of the COP path, mean velocity of the COP [MG1990], etc. Time-domain “area” measures include 95% confidence circle area which is the area of a circle with a radius equal to the one-side 95% confidence limit of the RD time series, 95% confidence ellipse area which is expected to enclose approximately 95% of the points on the COP path, etc. There are also time-domain “hybrid” measures. For example, sway area estimates the area enclosed by the COP path per unit of time [AH1980]. The mean frequency is the rotational frequency, in revolutions per second or Hz, of the COP if it had travelled the total excursions around a circle with a radius of the mean distance [FH1989]. The fractal dimension is a unitless measure of the degree to which a curve fills the metric space which it encompasses.

Apart from time-domain measures, there are also frequency-domain measures. A variety of qualitative and quantitative methods have been used to characterize the frequency distribution of the displacement of the COP [ID1983, TP1993], such as power spectral moments, total power, 50% power frequency, 95% power frequency, centroidal frequency, frequency dispersion, etc. There are also some statistical measures, like Romberg ratios, the phase plane parameter of Riley, etc.

It is worth pointing out that in 1981, the International Society of Posturography suggested the use of two COP-based measures, mean velocity of the COP and root mean square distance of the COP from origin, in their recommendations for standardizing force platform-based evaluation of postural steadiness [ID1983].

Since COP can be calculated from the pressure distribution under the user's feet obtained by the Balance Sensor of the present invention, all of the above COP-based measures can be adopted in the applications of the Balance Sensor. Moreover, pressure distribution has much more abundant information than a single COP position. With the pressure distribution under the user's feet, pedography analysis can be used. Pedography is a functional diagnostic tool, which can provide accurate, reliable information for the analysis of foot function and the diagnosis of foot pathologies. Foot deformities and malfunction can be detected during analysis of barefoot pressure distribution. This extra pathological information will greatly facilitate balance ability and fall assessment.

In addition, in the above COP-based measurements and assessments, COP can be replaced with center of gravity (COG). In this way, a series of COG-based measures can be created. Moreover, since the motion of the COG is the real physical motion of the human body and COP can be considered as the control of the human body in order to keep balance, a comparison of the variation of COP and COG can be used to analyze the balance control ability of the human body, which is a direct indicator of human balance ability and the degree of tendency to fall. As a result, a more accurate assessment is obtained.

The COP measurement, pedography analysis and COG measurement abilities of balancing can be integrated to develop fall assessment software. The core part of the software is a regression model generated through machine learning, which outputs the fall probability of the tester. This regression model is fused by two parts. One is based on a support vector machine. Those COP-based measurements, pedography analysis results and COG-based measurements are extracted and fed into this support vector machine. This support vector machine outputs a fall probability of the user or tester. Another part is based on use of a deep convolutional neural network, which will directly take the video data from the Balance Sensor as an input and output another fall probability of the tester. Then a weighted average of the two fall probabilities is taken from the support vector machine and the deep neural network as the final evaluation result of the fall assessment software.

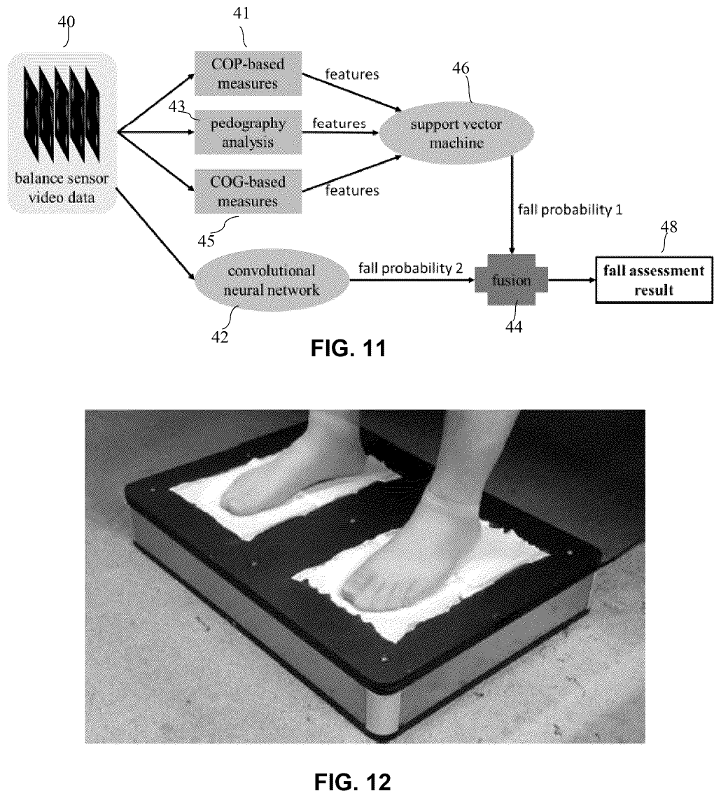

The diagram of the regression model is shown in FIG. 11. At step 40 the Balance Sensor video data is obtained. It is used to determine COP-based measurements at 41, pedography analysis at 43, COG-based measures at 45 and is also passed to convolutional neural network 42. The feature outputs of the COP, pedography and COG are combined in support vector machine 46, whose output is the fall probability 1. The output of convolutional neural network 42 is fall probability 2. Fall probabilities 1 and 2 are combined in fusion machine 44, whose output becomes the fall assessment result 48. All of the parameters in support of the vector machine 46, convolutional neural network 42 and the fusion weight 44 are trainable. In order to obtain this model, human experiment data is collected and labelled for training and testing as being data from normal people, old people and patients whose balance ability is affected by disease.

The assessment results are displayed on the screen 29 on the Balance Sensor and/or are announced by voice from a speaker, not shown. Furthermore, the results can also be transmitted via WiFi or Bluetooth to mobile devices 25 and/or other computers (not shown) for display and recording.

The rectangular box 22 of the sensing unit may have a size, e.g., of about 60×43×10 cm³, as shown in FIG. 12. For each of the two glass plates 14, the size is about 36×18×1 cm³. The two pieces of disposable latex sheet are shown in FIG. 12 on top of the two glass plates. The testers feet are shown on each latex sheet.

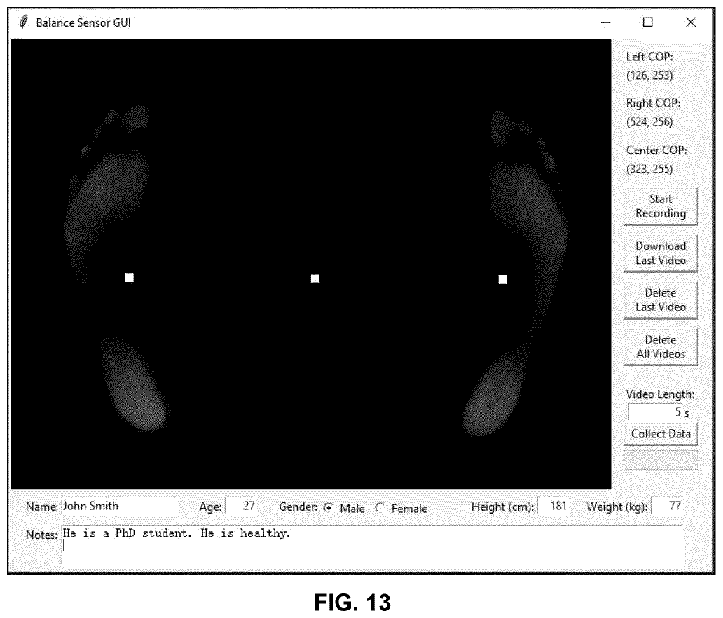

The graphical user interface (GUI) of the Balance Sensor is shown in FIG. 13. This GUI is mainly used for program setup and maintenance of the sensor, and can be run on a mobile device or PC connected with the Balance Sensor via WiFi. The interface can also be used to display the live stream of images captured by the camera 10. FIG. 13 shows the haptic image of tester's feet. The intensities of the pixels of the images are not identical due to the non-uniform tread force distribution under the user's or tester's feet. On the haptic image the 3 white points represent the pseudo centers of the left foot's pressure, the whole pressure distribution and right foot's pressure, respectively from left to right. The coordinates of these centers are shown on the top right corner of the GUI. The positions and coordinates of these centers also vary in the video live stream.

On the right side of the GUI of FIG. 13, apart from the coordinates of the 3 centers, there are some buttons used to control the camera. Using these buttons video can be recorded manually by controlling the start moment and stop moment. Alternatively, video duration can just be discretionarily set at a fixed value and the start of the collection of video data can be initiated. Further, the recorded video can be downloaded from the camera to a computer for further study. On the bottom of the GUI, there are several entries for inputting the user or testers' personal information, such as age, gender, height, weight, etc. After the “Collect Data” button is clicked, video will be recorded and downloaded into computer automatically. All the tester's personal information will be recorded into a separate csv file.

The procedure to turn on the Balance Sensor is as follows:
i. Place two clean disposable latex sheets on the glass plates on the top of the sensor.
ii. Turn on the sensor by stepping on it. A pressure switch (not shown) is provided under the upper surface for this purpose.
iii. The display or voice commands from a speaker (not shown) instruct the tester to stand still and the measurement starts in a couple of seconds.
iv. After a couple of minutes of taking measurements, the tester will be reminded of the end of the test by visual or audio commands.
v. The tester can step down from the sensor.
vi. The measurement data will be processed in the on-board microprocessor 20 and the results of the assessment are displayed on the screen 29 on the top of the sensor or they are announced by audio.
vii. The assessment results as well as the measurement data can be transmitted to mobile devices 25 or PCs via WiFi.
viii. The sensor will then turn off automatically after the testing.

Product setup procedure:
i. Launch the GUI from a mobile device or a PC connected with the product.
ii. Input the user's personal information, such as the user's name, age, weight, height, etc.
iii. Setup the assessment report, which can be a numerical value, qualitative grades, or color indicators in the form of a visual display and/or an audio announcement.
iv. Setup data records and transmit them.
v. Perform product self-calibration and testing.

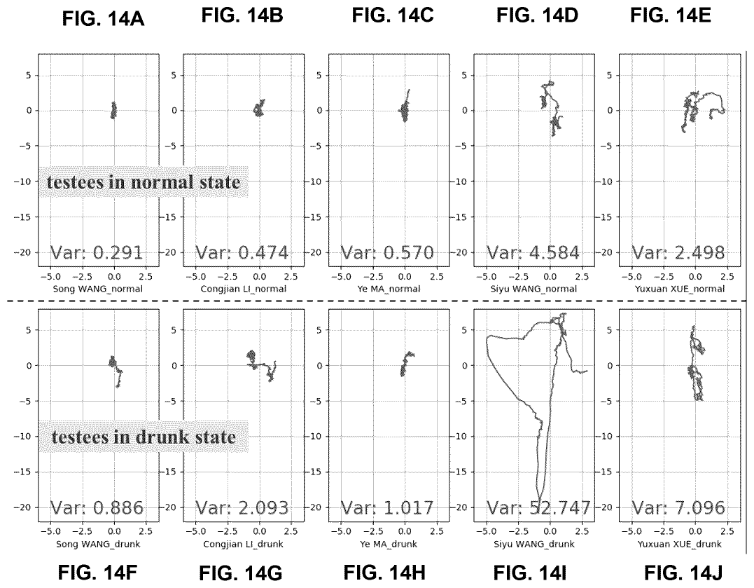

Experiments were conducted to test the measurement of human balancing using the sensor of the present invention. The test involved five recruited testers in total. Measurements of each of them were taken for 10 seconds with the Balance Sensor while the testers were in their normal states. The COP variations with time in a 2D plane for these 5 testers are shown in the first row of FIG. 14, i.e., FIGS. 14A-FIG. 14E. In comparison, data was collected when the testers were in abnormal states after extensive drinking of alcohol. The COP variations after drinking are shown in the second row of FIG. 14, i.e., FIGS. 14F-FIG. 14J. The comparisons of COP variation before and after drinking for these testers are shown in the columns. The red “Var” value indicates the variance or mean square of the distance of the COP from origin.

It can be seen in FIG. 14 that for every tester, after drinking, the “Var” value increased dramatically from the normal states as expected. This clearly indicates the decrease of balance ability after drinking. By observing the COP variation in a 2D plane, it can be directly found that the COP variation range increased after drinking, which means the sway of the human body increased. The experimental results demonstrate that the Balance Sensor can detect the slight change of human balancing ability and provide the basis for assessment of the risk of falling.

The cited references in this application are incorporated herein by reference in their entirety and are as follows:
F. O. Black, C. Wall III, H. E. Rockette Jr, and R. J. A. j. o. O. Kitch, “Normal subject postural sway during the Romberg test,” vol. 3, no. 5, pp. 309-318, 1982.
Y. Agrawal et al., “The modified Romberg Balance Test: normative data in US adults,” vol. 32, no. 8, p. 1309, 2011.
T. Michikawa, Y. Nishiwaki, T. Takebayashi, and Y. J. J. o. O. S. Toyama, “One-leg standing test for elderly populations,” vol. 14, no. 5, pp. 675-685, 2009.
J. M. Chandler, P. W. Duncan, and S. A. J. P. T. Studenski, “Balance performance on the postural stress test: comparison of young adults, healthy elderly, and fallers,” vol. 70, no. 7, pp. 410-415, 1990. 6.
S. W. Muir, K. Berg, B. Chesworth, and M. J. P. t. Speechley, “Use of the Berg Balance Scale for predicting multiple falls in community-dwelling elderly people: a prospective study,” vol. 88, no. 4, pp. 449-459, 2008.
S. Köpke and G. J. Z. f. G. u. G. Meyer, “The Tinetti test,” vol. 39, no. 4, pp. 288-291, 2006.
N. Rainville, “Effect of an implemented fall prevention program on the frequency of patient falls,” QRB. Quality review bulletin, vol. 10, no. 9, pp. 287-291, 1984.
L. J. Rapport et al., “Predictors of falls among right-hemisphere stroke patients in the rehabilitation setting,” Archives of physical medicine and rehabilitation, vol. 74, no. 6, pp. 621-626, 1993.
T. M. Steffen, T. A. Hacker, and L. J. P. t. Mollinger, “Age- and gender-related test performance in community-dwelling elderly people: Six-Minute Walk Test, Berg Balance Scale, Timed Up & Go Test, and gait speeds,” vol. 82, no. 2, pp. 128-137, 2002.
Y. J. C. M. A. J. Terekhov, “Stabilometry as a diagnostic tool in clinical medicine,” vol. 115, no. 7, p. 631, 1976.
R. A. Clark, A. L. Bryant, Y. Pua, P. McCrory, K. Bennell, and M. Hunt, “Validity and reliability of the Nintendo Wii Balance Board for assessment of standing balance,” Gait & posture, vol. 31, no. 3, pp. 307-310, 2010.
K. L. Johansen, R. D. Stistrup, J. Madsen, C. S. Schjøtt, and A. Vinther, “The timed up and go test and 30 second chair-stand test are reliable for hospitalized patients with stroke,” Physiotherapy, vol. 101, p. e918, 2015.
J. Goecke, “Effectiveness of the Stepping on Program in Fall Prevention Measured by the Four Stage Balance Test (FSBT),” 2017.
K. Berg, “Balance and its measure in the elderly: a review,” Physiotherapy Canada, vol. 41, no. 5, pp. 240-246, 1989.
J. G. Cwikel, A. V. Fried, A. Biderman, and D. Galinsky, “Validation of a fall-risk screening test, the Elderly Fall Screening Test (EFST), for community-dwelling elderly,” Disability and Rehabilitation, vol. 20, no. 5, pp. 161-167, 1998.
S. Whitney, M. Hudak, and G. Marchetti, “The dynamic gait index relates to self-reported fall history in individuals with vestibular dysfunction,” Journal of Vestibular Research, vol. 10, no. 2, pp. 99-105, 2000.
M. E. Tinetti, T. F. Williams, and R. Mayewski, “Fall risk index for elderly patients based on number of chronic disabilities,” The American journal of medicine, vol. 80, no. 3, pp. 429-434, 1986.
L. Wolfson, R. Whipple, P. Amerman, and J. N. Tobin, “Gait assessment in the elderly: a gait abnormality rating scale and its relation to falls,” Journal of gerontology, vol. 45, no. 1, pp. M12-M19, 1990.
J. M. VanSwearingen, K. A. Paschal, P. Bonino, and J.-F. Yang, “The modified Gait Abnormality Rating Scale for recognizing the risk of recurrent falls in community-dwelling elderly adults,” Physical Therapy, vol. 76, no. 9, pp. 994-1002, 1996.
J. M. Morse, R. M. Morse, and S. J. Tylko, “Development of a scale to identify the fall-prone patient,” Canadian Journal on Aging/La Revue canadienne du vieillissement, vol. 8, no. 4, pp. 366-377, 1989.
H. Maekawa, K. Tanie, K. Komoriya, M. Kaneko, C. Horiguchi, and T. Sugawara, “Development of a finger-shaped tactile sensor and its evaluation by active touch,” in Robotics and Automation, 1992. Proceedings., 1992 IEEE International Conference on, 1992: IEEE, pp. 1327-1334.
N. Nakao, M. Kaneko, N. Suzuki, and K. Tanie, “A finger shaped tactile sensor using an optical waveguide,” in Industrial Electronics Society, 1990. IECON′90., 16th Annual Conference of IEEE, 1990: IEEE, pp. 300-305.
S. Begej, “Planar and finger-shaped optical tactile sensors for robotic applications,” IEEE Journal on Robotics and Automation, vol. 4, no. 5, pp. 472-484, 1988, doi: 10.1109/56.20431.
S. Begej, “Fingertip-shaped optical tactile sensor for robotic applications,” in Robotics and Automation, 1988. Proceedings., 1988 IEEE International Conference on, 1988: IEEE, pp. 1752-1757.
S. Wang and N. Xi, “Recurrent Transfer Learning by Neural Network Regression for Human Balance Sensor Calibration,” in 2019 IEEE SENSORS, 2019: IEEE, pp. 1-4.
S. Wang and N. Xi, “Calibration of Haptic Sensors using Transfer Learning,” IEEE Sensors Journal, 2020.
S. Wang and N. Xi, “Sensing and Data Analysis for Assessing Human Balance Ability,” in 2018 IEEE 8th Annual International Conference on CYBER Technology in Automation, Control, and Intelligent Systems (CYBER), 2018: IEEE, pp. 807-812.
J. McAuley, J. Rothwell, and C. Marsden, “Frequency peaks of tremor, muscle vibration and electromyographic activity at 10 Hz, 20 Hz and 40 Hz during human finger muscle contraction may reflect rhythmicities of central neural firing,” Experimental brain research, vol. 114, no. 3, pp. 525-541, 1997.
T. E. Prieto, J. B. Myklebust, R. G. Hoffmann, E. G. Lovett, and B. M. Myklebust, “Measures of postural steadiness: differences between healthy young and elderly adults,” IEEE Transactions on biomedical engineering, vol. 43, no. 9, pp. 956-966, 1996.
M. Granat, C. Kirkwood, and B. Andrews, “Problem with the use of total distance travelled and average speed as measures of postural sway,” Medical & biological engineering & computing, vol. 28, no. 6, pp. 601-602, 1990.
A. Hufschmidt, J. Dichgans, K.-H. Mauritz, and M. Hufschmidt, “Some methods and parameters of body sway quantification and their neurological applications,” Archiv für Psychiatrie und Nervenkrankheiten, vol. 228, no. 2, pp. 135-150, 1980.
F. B. Horak, C. L. Shupert, and A. Mirka, “Components of postural dyscontrol in the elderly: a review,” Neurobiology of aging, vol. 10, no. 6, pp. 727-738, 1989.

I. DIRECTIONS, “Standardization in platform stabilometry being a part of posturography,” Agressologie, vol. 24, no. 7, pp. 321-326, 1983. While the invention is explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

1. A human balance sensor for assessing the risk of the user falling, comprising:
a transparent glass plate with flat upper and lower surfaces, and having a refractive index larger than that of air;
a latex sheet located on the top surface of the glass plate, during operation a foot of the standing user is placed on top of the latex sheet;
a light source located so as to inject light into the glass plate from its edge;
a high resolution camera located below the lower surface of the glass plate so as to capture light diffused from the glass plate when pressure is applied to the glass plate by the user's foot; and
whereby, based on the principle of Frustrated Total Internal Reflection (FTIR), when user stands with his foot on the glass plate (a) the latex sheet is pressed onto the upper surface of the glass plate, (b) a condition of total internal reflection is eliminated at the pressure locations due to the foot, and (c) diffused reflection of the light passes from the bottom surface of the glass plate and is focused onto an image plane of the camera to form a haptic image of the contact area of the foot with different pixel intensities based on different pressures from the foot at different locations on the glass plate.

2. The human balance sensor of claim 1 wherein the light source is an LED light source.

3. The human balance sensor of claim 2 wherein the LED light source is a strip of red LED lights located about the periphery of the glass.

4. The human balance sensor of claim 1 wherein the camera records a series of haptic images over a period of time; and
further including a microprocessor that analyzes the changes in the series of haptic images and determines human balance ability based thereon.

5. A human balance sensor for assessing the risk of the user falling, comprising:
a housing;
two transparent glass plates with flat upper and lower surfaces, and having refractive indices larger than that of air, said glass plates being located side-by-side on a top of the housing and spaced from each other by about the spacing of the feet of a standing human;
latex sheets, one located on the top surface of each of the glass plates, during operation the feet of the standing user are placed on top of the respective latex sheets;
a light source located so as to inject light into each of the glass plates from their edges;
a high resolution camera located below the lower surface of the glass plates so as to capture light diffused from the glass plates when pressure is applied to the glass plates; and
whereby, based on the principle of Frustrated Total Internal Reflection (FTIR), when user stands with his feet on the glass plates (a) the latex sheets are pressed onto the upper surfaces of the respective glass plates, (b) a condition of total internal reflection is eliminated at the pressure locations due to the feet, and (c) diffused reflection of the light passes from the bottom surfaces of the glass plates and is focused onto an image plane of the camera to form a haptic image of the contact area of the feet with different pixel intensities based on different pressures from the feet at different locations on the glass plates.

6. The human balance sensor of claim 5 wherein the camera records a series of haptic images over a period of time; and
further including a microprocessor that analyzes the changes in the series of haptic images and determines human balance ability based thereon.

7. The human balance sensor of claim 6 wherein the camera has a frame rate of about 30 fps and a resolution of about 1920×1440.

8. The human balance sensor of claim 7 wherein the camera has the ability to wirelessly transmit images to another computing device.

9. The human balance sensor of claim 7 further including a display on the upper surface of the housing for displaying fall assessment results as human balance ability.

10. The human balance sensor of claim 6 wherein the microprocessor analyzes the changes in the series of haptic images and determines human balance ability based on measurement of different coordinates of the center of pressure (COP) over time.

11. The human balance sensor of claim 10 wherein the COP measurements include at least one of
time-domain “distance” measurements of the mean distance of the COP from origin, root mean square distance of the COP from the origin, total length of the COP path and mean velocity of the COP;
time-domain “area” measurements of 95% confidence circle area, 95% confidence limit of the RD time series and 95% confidence ellipse area;
time-domain “hybrid” measurements of sway area estimates, the mean rotational frequency and the fractal dimension; and
frequency-domain measurements of power spectral moments, total power, 50% power frequency, 95% power frequency, centroidal frequency and frequency dispersion.

12. The human balance sensor of claim 6 wherein the microprocessor analyzes the changes in the series of haptic images and determines human balance ability based on pedography analysis.

13. The human balance sensor of claim 6 wherein the microprocessor analyzes the changes in the series of haptic images and determines human balance ability based on different coordinates of a series of center of gravity (COG) based measurements over time.

14. The human balance sensor of claim 6 wherein the microprocessor analyzes the changes in the series of haptic images and determines human balance ability based on a regression model that integrates COP measurement, pedography analysis and COG measurement;
wherein the regression model is fused by two parts,
one of which is based on the COP-based measures, pedography analysis results and COG-based measures extracted from the images and fed into a support vector machine, which outputs a first fall probability of the tester, and
the second of which is based on a deep convolutional neural network, which will directly take the video data from the balance sensor as an input and outputs a second fall probability of the tester; and
a weighted average of the first and second fall probabilities is taken from the support vector machine and the deep neural network as the final evaluation result of the fall assessment.

15. The human balance sensor of claim 6 further including means for manually extracting certain features are from the haptic images prior to the microprocessor analyzing the changes in the series of haptic images.

16. The human balance sensor of claim 6 further including means for training a deep learning algorithm, such as a 3D convolutional neural network (CNN), to generate a classification model prior to the microprocessor analyzing the changes in the series of haptic images.

17. The human balance sensor of claim 15 further including means for training a deep learning algorithm, such as a 3D convolutional neural network (CNN), to generate a classification model after the manual extraction and prior to the microprocessor analyzing the changes in the series of haptic images.

18. The human balance sensor of claim 6 wherein the microprocessor analysis is based on a model of the human body that comprises multiple differential equations associated with the pressure distribution variation process under the feet of the user, and the analysis is based on solution of the equations to obtain detailed body motion processes.

19. The human balance sensor of claim 18 wherein the microprocessor solves the differential equations based on an algorithm derived from a Generative Adversarial Tri (GAT) model.

20. The human balance sensor of claim 18 wherein the microprocessor solves the differential equations by a Generative Adversarial Tri-model (GAT) approach that combines an analytical approach with a neuro network to numerically solve nonlinear ordinary differential equations with non-initial conditions as follows:
initialize the neural network randomly or by an approximate solution;
train a model with the Euler loss function of the Runge-Kutta loss function until convergence to obtain the numerical solutions;
determine if convergence has been reached, if not the adjust the current outputs of the neural network to satisfy definite conditions and retrain the model;
if convergence has been reached, end the process with the current results.

21. The human balance sensor of claim 20 wherein the neural network is initialized with an approximate solution wherein the nonlinear terms in the equations are first discarded; and determining the solution through the finite difference method with the help of definite conditions.

22. The human balance sensor or claim 1 wherein the light given by the light source is invisible or more preferably an infrared light.
